(19) United States
(12) Patent Application Publication
(10) Pub. No.: US 2012/0134466 A1
(43) Pub. Date: May 31, 2012

(54) GALACTOGRAPHY PROCESS AND MAMMOGRAPH

(76) Inventors: Serge Muller, Guyancourt (FR); Silvie Puong, Paris (FR)

(21) Appl. No.: 13/300,416

(22) Filed: Nov. 18, 2011

(30) Foreign Application Priority Data
Nov. 26, 2010 (FR) 1059795

(51) Int. Cl. A61B 6/00

(52) U.S. Cl. 378/37

(57) ABSTRACT

A galactography process in a mammograph for characterizing galactophorous ducts of a breast of a patient in which a contrast product has been previously injected is provided. The process comprises: emitting X-rays to the breast of a patient; acquiring at least one first mammographic image with X-rays having a first energy; acquiring at least one second mammographic image with X-rays having a second energy; and processing the at least one first image and the at least one second image to produce an image of the concentration of the contrast product in the breast, wherein the first energy is different than the second energy.

### BACKGROUND OF THE INVENTION

1. Field of the Invention

The disclosure relates to a galactography process in a mammograph, and a mammograph for executing the process.

2. Description of the Prior Art

Galactography is a radiological examination of the breast of a patient. Apart from soft tissue, such as adipose and fibrous tissue, the breast of a patient comprises an arborescent network of ducts, called galactophorous ducts, which convey milk to the nipple. These ducts terminate at the nipple via galactophorous orifices. There are typically as many as fifteen to twenty galactophorous orifices. Galactography enables said galactophorous ducts to be viewed.

This type of examination is indicated particularly in the event of flow from the breast outside the periods of lactation. This examination allows pre-operatory evaluation of the nature, location and extent of lesions, especially cancerous, capable of causing said nipple flow.

Galactography techniques have evolved little over time. In particular, only two major evolutions in galactography techniques have occurred in the past. A first evolution consisted of converting images on classic radiological film to digital images. Despite this, the image of galactophorous ducts opacified by prior injection of a contrast product superposing soft tissue on the image, it can be difficult for the practitioner to locate or analyse the characteristics of the smallest ducts. A second evolution consisted of creating a subtracted galactography process using the combination of two images acquired before and after injection of a contrast product, to better characterize the galactophorous ducts.

Document FR2816822 discloses a galactography process according to the prior art. In the process, a practitioner detects the galactophorous orifice at the origin of flow at the level of the nipple. The practitioner then dilates said galactophorous orifice with a needle or a canula in foam to enable later injection of a contrast product. A first step consists of acquiring a first image of the breast of the patient in a compressed state, without contrast product. A contrast product (ex: iodine), attenuating to X-rays, is injected into the galactophorous ducts by this needle or canula. The process comprises a second step consisting of acquiring a second image of the breast of the patient in a compressed state and comprising the contrast product. Finally, the process comprises a subtraction step, partial or complete, of the first image relatively or the second image, or inversely.

As it is understood, the first image comprises both fibrous or adipose tissue and galactophorous ducts. The second image comprises, apart from fibrous or adipose tissue, galactophorous ducts, which have been opacified in the image by injection of a contrast product. Thus, subtraction of images retains in the image only the part opacified by the contrast product, that is, the galactophorous ducts.

If this process to some degree characterizes the galactophorous ducts, it nevertheless has disadvantages. The contrast product is injected between two taking of mammographic images events, which perturbs the process of taking images and prolongs its duration. Also, this process restricts the breast of the patient to remain under compression for a longer duration, which is a source of discomfort for the patient. Furthermore, the complexity of carrying out this process and its constraints for clinical use are such that, to date, there has not been any practical application of it marketed.

In light of the above, the medical profession has tended to ignore galactography, to the benefit of alternative techniques. Evolution and improvement of galactography processes known to date should therefore be proposed.

### BRIEF SUMMARY OF THE INVENTION

The invention proposes eliminating the above disadvantages.

In an embodiment of the present invention, a galactography process in a mammograph for characterizing galactophorous ducts of a breast of a patient in which a contrast product has been previously injected is provided. The process comprises: emitting X-rays to the breast of a patient; acquiring at least one first mammographic image with X-rays having a first energy; acquiring at least one second mammographic image with X-rays having a second energy; and processing the at least one first image and the at least one second image to produce an image of the concentration of the contrast product in the breast, wherein the first energy is different than the second energy.

In another embodiment of the present invention, a mammograph is provided. The mammograph comprises: a source of X-rays configured to emit X-rays to the breast of a patient in which a contrast product has been previously injected; a detector of X-rays positioned to detect X-rays from the source; a control unit configured to control acquisition of at least one first mammographic image with X-rays having a first energy and acquisition of at least one second mammographic image with X-rays having a second energy, wherein the first energy is different than the second energy; and a processing unit configured to process the at least one first image and the at least one second image to produce an image of the concentration of the contrast product in the breast.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic view of a mammograph according to an embodiment of the invention;

FIG. 2 is a schematic view a process according to an embodiment of the invention;

FIG. 3 is a schematic view of an image of the breast in which galactophorous ducts comprise a contrast product, made with X-rays having a first energy;

FIG. 4 is a schematic view of an image of the breast in which galactophorous ducts comprise a contrast product, made with X-rays having a second energy;

FIG. 5 is a schematic view of an image after processing of images of FIGS. 3 and 4; and

FIG. 6 is a schematic view of an image after processing of images of FIGS. 3 and 4.

### DETAILED DESCRIPTION OF THE INVENTION

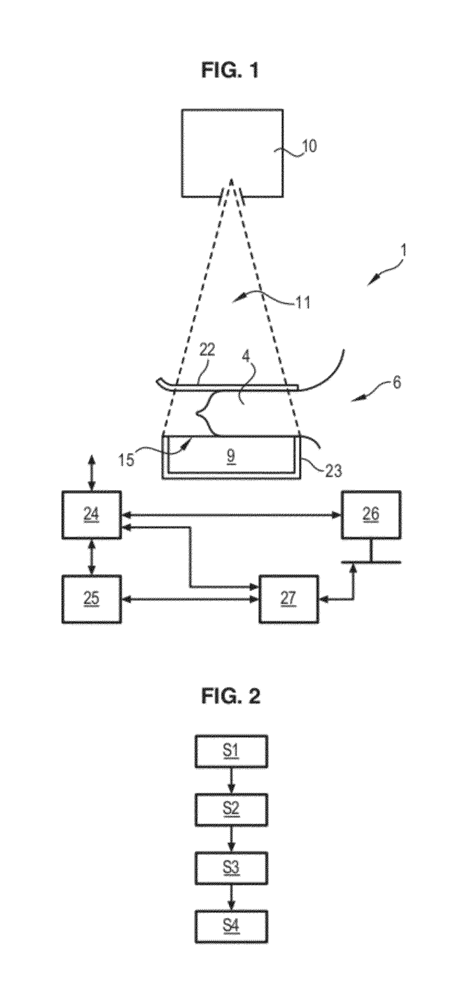

FIG. 1 schematically illustrates a mammograph 1 according to an embodiment of the invention. The mammograph 1 comprises a source 10 of X-rays and an X-ray detector 9. The source 10 of X-rays is capable of emitting X-rays 11 to the detector 9, for taking mammographic images of a patient 6.

The mammograph 1 comprises an upper plate 22, called a compression pad, and a lower block 23. The upper plate 22 is mobile in vertical translation to compress the breast 4 of the patient 6 against the lower block 23. Alternatively, or in addition, the lower block 23 is mobile to enable compression of the breast 4.

The detector 9 comprises a detection surface 15 turned to the source of X-rays, under the breast 4 of the patient 6. The detector 9 is for example a semi-conductor image sensor or a CCD sensor. These types of detectors are given by way of non-limiting examples.

The X-rays emitted by the source 10 encounter the breast 4 of the patient 6, and the detector 9 sense the X-rays transmitted by the breast to take a mammographic image.

It is possible to provide an anti-diffusing grid between the source 10 and the detector 9, comprising absorption blades (“septa”) opaque to X-rays, which filter unwanted rays diffused by the breast of the patient 6. Alternatively, or in addition, collimation between the source 10 and the detector 9 can be provided.

The mammograph 1 may comprise a control unit 24, a storage unit 25, a display unit 26, and a processing unit 27. The control unit 24 controls acquisition by fixing several emission parameters of X-rays by the source 10. The control unit 24 likewise controls displacement of the source 10 and/or of the detector 9, as well as their relative positions. The control unit 24 is typically a microcomputer and/or a processor.

The storage unit 25 may be connected to the control unit 24 for recording parameters and images acquired. In one embodiment the storage unit 25 may be located inside the control unit 24. In an alternate embodiment, the storage unit 25 may be located or outside the control unit 24. The storage unit 25 can be formed by a hard drive or SSD, or any other means of removable and rewritable storage (USB flash drives, memory cards etc.). The storage unit 25 can especially be ROM/RAM memory of the control unit 24, a USB flash drive, a memory card, memory of a central server, etc.

The display unit 26 may be connected to the control unit 24 for displaying images acquired and/or information on the control parameters of acquisition. The display unit 26 can be for example a computer screen, a monitor, a flat screen, plasma screen or any other type of display device of known type. Such a display unit 26 allows a practitioner to view and control acquisition of images by the mammograph. The mammograph further may comprise means of interaction for a practitioner, of keyboard type.

The mammograph 1 further comprises a processing unit 27, capable of processing the images according to the galactography process described later. The processing unit 27 may be a microcomputer and/or processor for communicating with the control unit 24, the storage unit 25 and the display unit 26.

It is understood that the functional clipping of the different control, display, storage and processing units which have just been described can be different according to embodiments and needs.

In one embodiment, the source 10 and/or the detector 9 are advantageously mobile. In one embodiment, the source 10 and/or the detector 9 can be shifted relative to one another in different relative angular positions for taking three-dimensional mammographic images by tomosynthesis. The relative angular displacement can consist for example of displacement of the source 10 on an arc of a circle, or on a line, or any other trajectory adapted to need. This produces a series of images of the breast, corresponding to a series of projections of the breast according to different angles. In general, the relative angular displacement has limited amplitude (between ±7° and ±60°, these values being non limiting).

A set of images describing the volume of the breast using image-processing algorithms known to the person skilled in the art can be reconstructed from this set of images. The invention is likewise applicable to mammographs of scanner type dedicated to the imaging of the breast.

The source 10 and the detector 9 may be set in rotation around the breast of the patient according to an angle generally between 0 and 360 degrees, for taking three-dimensional mammographic images, as in a scanner.

Different embodiments of the galactography process according to the present invention will now be described, using one or the other of the embodiments of the mammograph described earlier.

Prior to the galactography process, a practitioner marks the galactophorous orifice at the origin of the flow at the level of the nipple. The galactophorous orifice is then dilated with a needle or a canula in foam. Once the orifice is sufficiently dilated, a contrast product, which is attenuating for X-rays, is injected by this needle or canula. It can be, for example, a hollow needle of a diameter of the order of 1.0 mm, or any other instrument used conventionally in galactography.

The contrast product is generally a contrast product of hydrosoluble iodine. The galactophorous orifice may be stoppered by means of a wax-based stopper.

The breast 4 of the patient 6 is then positioned on the lower block 23 comprising the detector 9, then the breast is put under compression using the compression pad 22. Compression is sufficient for the breast of the patient to be immobilised during the examination so as to avoid a blurred image, but to allow the contrast product to circulate. At this stage, one or more galactophorous ducts 5 of the breast 4 of the patient 6 include a previously injected contrast product.

As illustrated in FIG. 2, the galactography process comprises a step S1 consisting of emitting X-rays to the breast 4 of the patient 6 for taking mammographic images 7, said breast 4 comprising galactophorous ducts 5 into which a contrast product 8 has been previously injected. The X-rays are emitted by the source 10 of X-rays of the mammograph 1.

The process likewise comprises a step S2 consisting of taking at least one first mammographic image with X-rays having a first energy E₁, and a step S3 consisting of taking at least one second mammographic image with X-rays having a second energy E₂. The first energy E₁ is greater than the second energy E₂, or inversely, the second energy E₂ being greater than the first energy E₁ (E₁>E₂ or E₁<E₂).

The X-rays passing through the breast of the patient are collected by the detector 9, producing at least the first and the second images. An example of a first image is represented in FIG. 3. The galactophorous ducts 5 comprising the contrast product 8 are represented here schematically by grooves, to make the figure clear to read. The image likewise comprises soft tissue 20, such as adipose, and/or fibro-glandular tissue.

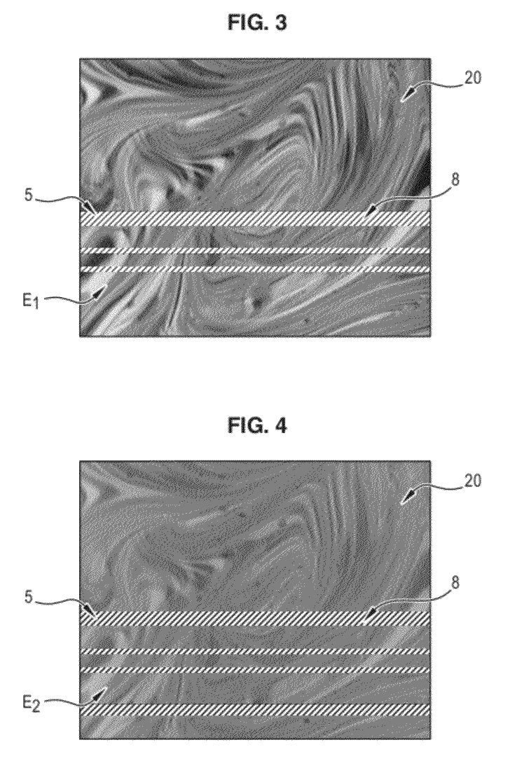

An example of a second image is represented in FIG. 4, specifically the case wherein Eᵢ<E₂. As is evident, the contrast product 8 is more visible in the second image. On the contrary, soft tissue exhibits lower contrast in the second image than in the first image.

The process further comprises a step S4 consisting of processing the first and the second images to produce an image of the concentration of the contrast product 8 in the breast 4, and thus characterize the galactophorous ducts 5 of said breast 4. The concentration can be surface concentration (concentration generally expressed in mg/cm²), or volumetric (concentration generally expressed in mg/cm³), in the case of three-dimensional reconstruction.

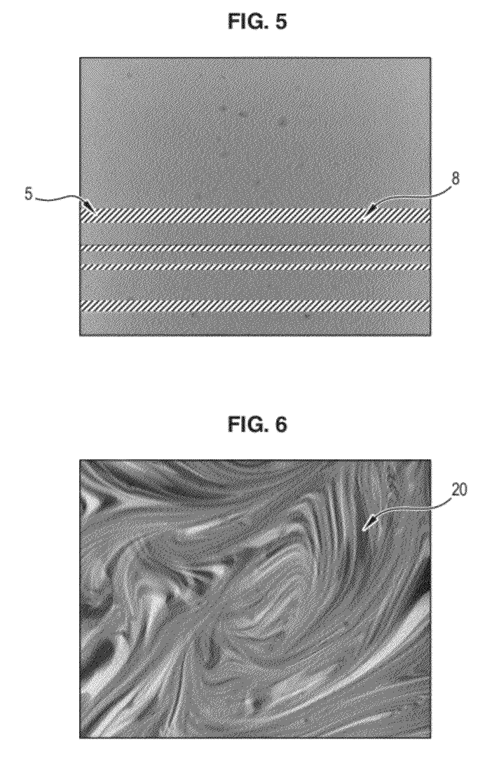

FIG. 5 is a schematic representation of the type of image which can be obtained in the process according to embodiments of the present invention. This clearly distinguishes the contrast product 8, which materializes the presence of the galactophorous ducts 5. As is evident, the processing eliminated the soft tissue of the breast in the image.

FIG. 6 is an image in which the contrast product 8 has been eliminated, showing only the soft tissue of the breast of the patient. As it is known, an image is represented by spatial distribution of intensities, which are most often grey levels. The processing step S4 comprises mathematical processing of the intensities in the first and second images, described later. As explained hereinafter, various processing techniques can be used.

In one embodiment, the process can be applied in the case of taking three-dimensional images. In the case of tomosynthesis, the process comprises a step consisting of producing relative angular displacement between the source 10 and the detector 9, in different relative angular positions. For each relative angular position, the process comprises a step consisting of taking at least one first mammographic image with X-rays having a first energy E₁, and at least one second mammographic image with X-rays having a second energy E₂, the first energy E₁ being greater than the second energy E₂, or inversely, the second energy E₂ being greater than the first energy E₁.

The process likewise comprises a step consisting of processing the first and second images for each relative angular position to produce a plurality of images of the concentration of the contrast product 8 in the breast 4.

Another step consists of applying a three-dimensional algorithm reconstruction, known to the person skilled in the art, (for example “Filter Back Projection (FBP)” or “Simultaneous Algebraic Reconstruction Technique (SART) algorithms”) to the plurality of images of the concentration of the product taken in each position to produce an image of the volumetric concentration of the product 8 in the breast. This time, access is gained to concentration in three dimensions (volumetric) of the product 8 in the breast.

Similarly, in the case of viewing by scanner type, the process comprises a step consisting of producing simultaneous rotation of the source 10 and of the detector 9 around the breast 4 of the patient 6, in various positions between 0 and 360° around the breast 4. For each of said positions, the process comprises a step consisting of taking at least one first mammographic image with X-rays having a first energy E₁, and at least one second mammographic image taken with X-rays having a second energy E₂, the first energy E₁ being greater than the second E₂, or inversely, the second energy E₂ being greater than the first energy E₁.

The process likewise comprises a step consisting of processing the first and the second images for each position to produce a plurality of images of the concentration of the contrast product 8 in the breast 4.

Another step consists of applying a three-dimensional algorithm reconstruction to the plurality of images of the concentration of the product made in each position to produce an image of the volumetric concentration of the product 8 in the breast. Access therefore is likewise gained to concentration in three dimensions of the product 8 in the breast. The advantage of taking images of scanner type relative to tomosynthesis is the resulting precision, especially by obtaining a substantially isotropic volume of data.

In the two cases above, it is clear that three-dimensional reconstruction can be applied to the images originating from processing low and high-energy images (step S4), or inversely, be applied separately to low and high-energy images, before processing according to step S4. In the latter case, the processing (step S4) for determining the concentration of the product is then applied to three-dimensional images.

Typical values for the lowest energy (E₁ or E₂ according to case) are located around 20 keV, whereas typical values for the highest energy (E₁ or E₂ according to case) are located around 34 keV. These values are given by way of non-limiting example. These are values located on either side of the ionization energy of iodine (electron K). Above this ionization energy, iodine exhibits an attenuation peak to X-rays, which make it highly visible in the image. Below this ionization energy, iodine is less visible. It is understood that it is not obligatory to select energies on either side of this ionization energy.

Step S2 consisting of taking the first mammographic image can be made via emission of X-rays at the first energy E₁. This can be obtained by controlling the emission parameters of the source of X-rays (accelerating voltage between the anode and the cathode of the source of X-rays, electric intensity applied to the filament of the cathode, etc.).

In the same way, step S3 consisting of taking the second mammographic image can be conducted via the emission of X-rays at the second energy E₂. Alternatively, taking the first and second mammographic images comprises the emission of X-rays in an energy range comprising the first and second energies E₁, E₂, and filtering of the energy of the rays by means of filters arranged at the outlet of the radiation source. It is understood that this is generalisable to N energies E₁, . . . E_{N}.

Alternatively, or in addition, taking the first and the second mammographic images comprises the emission of X-rays in an energy range comprising the first and second energies E1, E2, and the detection of the X-rays by an X-ray detector 9 of the mammograph, configured to discriminate the energy of said X-rays. In this case, the detector 9 can discriminate the energy or the energy range of X-rays emitted by the source 10 to the detector 9, for taking mammographic images at different emission energies of X-rays. So, it is the detector 9 which plays the role of filter as a function of the energy of the X-rays. This type of detector 9 is generally based on photon-counting technology, comprising the capacity to discriminate the energy of photons and creation of an electric signal correlated to the energy of said photons. At least one first image at the energy E₁ and at least one second image at the energy E₂ are finally produced.

The process may comprise the steps consisting of taking a plurality of images with X-rays having a different energy (E₁, E₂, . . . E_{N}) from one image to the other. This gives, for example, N images with N different energies. Consequently, the different types of tissue present in the breast can be better characterized and the concentration of the contrast product can therefore be deduced with greater precision. Alternatively, several images at the same energy can be obtained.

Various techniques for processing of images taken by the mammograph 1 will now be described, resulting in the concentration of the contrast product in the image.

The general principle of this processing step S4 is based on the fact that matter presents a different attenuation coefficient according to the type of matter, the concentration of matter, and the energy of the X-rays. Therefore, by taking images at different energies of X-rays, the tissue which is not generally useful in galactography, such as the soft tissue, can be eliminated from the image by appropriate processing and solely the contrast product can be kept, which characterizes the galactophorous ducts. This results in an image of the concentration of the contrast product, which has information on the distribution of the galactophorous ducts, their dimensions, their arborescence, the quality of said ducts, etc.

This mathematical processing is based more precisely on the following considerations. The following elements are especially visible in the images taken by the mammograph: soft tissue 20, comprising a variety of different tissue such as adipose and fibro-glandular tissue, as well as the contrast product 8 introduced to the galactophorous ducts 5. The contrast product 8 and soft tissue 20 appear in the image made with low-energy X-rays, (for example energy E1, in FIG. 3). But the contrast product 8 will be less visible than in an image made with high-energy X-rays. Also, it is evident that the contrast between the different types of soft tissue 20 is high.

In the image made with high-energy X-rays, (for example the energy E₂>Eᵢ, in FIG. 4) the contrast product 8, and soft tissue 20 could likewise be distinguished. In this case, the contrast product 8 will be highly visible, given that the contrast product 8 will show a high-energy attenuation peak. On the contrary, the contrast between the different types of soft tissue will be lower.

Making a mathematical combination of the intensities of these images can selectively “eliminate” soft tissue 20 or the contrast product 8 from the image, as shown in FIGS. 5 and 6. Various mathematical models exist.

Since attenuation of X-rays by matter can be described by the Beer-Lambert law, if it is supposed that incident X-rays have a perfectly monochromatic energy, the result is a relation of type:

I=I₀exp(−μ·L),

with I being the intensity of the X-rays perceived by the detector, I₀ the intensity of X-rays emitted by the source, μ the attenuation coefficient of matter present in the breast, and L the thickness traversed of the image material supposed to be uniform for the sake of simplification. Access is then gained to the radiological thickness μ·L by applying a transformation logarithm μ·L=Ln(I₀/I) on the intensities I of the image.

When two images are acquired at low and high energies E₁, E₂, this relation accesses measurements of the grey levels G_{E1} and G_{E2} in the image, respectively by transformation logarithm on the intensities of images acquired at low and high energies E₁, E₂. The result is a system of two linear equations whereof the two unknown quantities are the thicknesses Lᵢ and Lₜ of the contrast product and of the soft tissue respectively.

Ln(G_{E1})=μᵢ(E₁)·Lᵢ+μₜ(E₁)·Lₜ

Ln(G_{E2})=μᵢ(E₂)·Lᵢ+μₜ(E₂)·Lₜ

Knowing the values of the attenuation coefficients μᵢ of the contrast product and μₜ of the soft tissue for energies E₁ and E₂ respectively, this system of equations is easy to resolve, and accesses the concentration of the contrast product in the breast (directly linked to the thickness “Lᵢ” of the contrast product). In the case of an image in two dimensions, this is surface concentration (example of unit: mg/cm²). In the case of an image in three dimensions (tomosynthesis, scanner), this is volumetric concentration (example of unit: mg/cm³). In a more refined model, it is supposed that the energy of X-rays is not perfectly monochromatic, leading to a non-linear system of equations.

A method of resolution consists of finding a function ƒ calibrated so that:

x_{product}=ƒ(ln(G_{E1}),ln(G_{E2})),

where x_{product} is the thickness of contrast product, G_{E1} the level of grey (intensity) of the low-energy image E₁, and G_{E2} the grey levels (intensity) of the high-energy image E₂.

A function linking the thickness or equally the concentration of the product to the logarithms of the intensities of the first and second images, or of the plurality of images of different energies should therefore be determined. This function can be determined by digital simulation or by experimentation.

In general, calibration or modelling by digital simulation for different known values of concentration and distribution of the contrast product, and different known values of concentration and distribution of soft tissue enables having reference values, from which the function ƒ is determined. In fact, knowing the grey level caused by known distribution of the concentration of the contrast product and known distribution of soft tissue can help to estimate the most adequate function ƒ.

In an one embodiment, a quadratic approximation of functions ln(G_{E1}) and ln(G_{E2}) is used, of type:

x_{produit}=a₀+a₁ ln G_{E1}+a₂ ln G_{E2}+a₃(ln G_{E1})²+a₄(ln G_{E2})²+a₅ ln G_{E1}·ln G_{E2}

In this case, the coefficients aᵢ are to be estimated.

In general, as understood by the person skilled in the art, it is possible to apply the earlier described instances of processing to the event where a plurality of images at different energies (E₁, E₂, . . . E_{N}) is taken. Function ƒ is then determined, such that: x_{product}=ƒ(ln(G_{E1}), . . . , ln(G_{EN})). As it is understood, the results of the concentration of the contrast product are more precise when there are several images at different energies.

Finally, embodiments of the invention relate to a computer program comprising product instructions for controlling the mammograph for carrying out the process earlier described. This program executes the instructions for controlling the emission of X-rays, producing images at different energy, and appropriate processing of the resulting images, according to the process of the invention. The computer program may be loaded in the processing unit of the mammograph.

Because of the process according to the invention, galactophorous ducts can be better characterized, thus largely improving current galactography processes. In particular, the invention better differentiates the galactophorous ducts from soft tissue of the breast, by reducing or eliminating the superposition of soft tissue and opacified galactophorous ducts, crucial for a practitioner.

Also, the galactography process according to embodiments of the present invention is faster, and requires no injections when images of the breast of the patient are being taken. This results especially in a galactography process simpler to implement.

Finally, embodiments of the present invention propose a galactography process for obtaining a finer vision of the distribution of tissue and ducts in the breast.

Embodiments of the present invention therefore provide a major advantage for practitioners and people utilizing galactography techniques. It is evident that galactography tended to be ignored by the medical profession for its lack of precision and performance. The process according to the embodiments of the present invention substantially improve galactography processes known to date.

What is claimed is:

1. A galactography process in a mammograph for characterizing galactophorous ducts of a breast of a patient in which a contrast product has been previously injected, the process comprising:
emitting X-rays to the breast of a patient;
acquiring at least one first mammographic image with X-rays having a first energy;
acquiring at least one second mammographic image with X-rays having a second energy; and
processing the at least one first image and the at least one second image to produce an image of the concentration of the contrast product in the breast, wherein the first energy is different than the second energy.

2. The process of claim 1, wherein:
acquiring at least one first mammographic image comprises emitting X-rays at a first energy, and
acquiring at least one second mammographic image comprises emitting X-rays at a second energy.

3. The process of claim 1, wherein acquiring at least one first mammographic image and acquiring at least one second mammographic image comprises emitting X-rays in an energy range comprising the first and the second energies and the detection of X-rays by an X-ray detector of the mammograph, configured to discriminate the energy of said X-rays.

4. The process of claim 1, comprising acquiring a plurality of images, wherein each image is acquired with an energy that is different from each of the other images.

5. The process of claim 1, wherein the mammograph comprises a source of X-rays and a detector of X-rays, the process further comprising:
producing relative angular displacement between the source and the detector in different relative angular positions,
wherein acquiring at least one first mammographic image with X-rays having a first energy and acquiring at least one second mammographic image with X-rays having a second energy is performed for each relative angular position; and
wherein processing the at least one first image and the at least one second image is performed for each relative angular position to produce a plurality of images of the concentration of the contrast product in the breast.

6. The process of claim 5, further comprising applying a three-dimensional reconstruction algorithm to the plurality of images of the concentration of the contrast product to produce an image of the volumetric concentration of the contrast product in the breast.

7. The process of claim 1, wherein the mammograph comprises a source of X-rays and a detector of X-rays, the process further comprising:
producing simultaneous rotation of the source and of the detector around the breast of the patient in diverse positions between 0 and 360°, wherein acquiring at least one first mammographic image with X-rays having a first energy and acquiring at least one second mammographic image with X-rays having a second energy is performed for each position; and
wherein processing the at least one first image and the at least one second image is performed for each position to produce a plurality of images of the concentration of the contrast product in the breast.

8. The process of claim 7, further comprising applying a three-dimensional reconstruction algorithm to the plurality of images of the concentration of the contrast product to produce an image of the volumetric concentration of the contrast product in the breast.

9. The process of claim 1, processing the at least one first image and the at least one second image to produce an image of the concentration of the contrast product in the breast comprises determining a function linking the concentration of the product to the logarithms of the intensities of the at least one first image and the at least one second image.

10. A mammograph comprising:
a source of X-rays configured to emit X-rays to the breast of a patient in which a contrast product has been previously injected;
a detector of X-rays positioned to detect X-rays from the source;
a control unit configured to control acquisition of at least one first mammographic image with X-rays having a first energy and acquisition of at least one second mammographic image with X-rays having a second energy, wherein the first energy is different than the second energy; and
a processing unit configured to process the at least one first image and the at least one second image to produce an image of the concentration of the contrast product in the breast.

11. The mammograph of claim 10 further comprising a storage unit operatively connected to the control unit, wherein recording parameters and acquired images are stored in the storage unit.

12. The mammograph of claim 10 further comprising a display unit, wherein acquired images and/or control parameter information are displayed by the display unit.

13. The mammograph of claim 10, wherein the at least one first mammographic image and the at least one second mammographic image are acquired at an energy range comprising the first and the second energies, and wherein the detector is configured to discriminate the energy of the X-rays.

14. The mammograph of claim 10, wherein the control unit is configured to acquire of a plurality of images, wherein each image is acquired with an energy that is different from each of the other images.

15. The mammograph of claim 10, wherein the control unit is further configured to produce relative angular displacement between the source and the detector in different relative angular positions and the control unit is further configured to control acquisition of the at least one first mammographic image and the at least one second mammographic image with X-rays having a second energy for each relative angular position, and
wherein the processing unit is further configured to process the at least one first image and the at least one second image for each relative angular position.

16. The mammograph of claim 15, wherein the processing unit is further configured to apply a three-dimensional reconstruction algorithm to the plurality of images of the concentration of the contrast product to produce an image of the volumetric concentration of the contrast product in the breast.

17. The mammograph of claim 10, wherein the control unit is further configured to produce simultaneous rotation of the source and of the detector around the breast of the patient in diverse positions between 0 and 360° and wherein the control unit is further configured to control acquisition of the at least one first mammographic image and the at least one second mammographic image for each position; and
wherein the processing unit is further configured to process the at least one first image and the at least one second image for each position.

18. The mammograph of claim 17, wherein the processing unit is further configured to apply a three-dimensional reconstruction algorithm to the plurality of images of the concentration of the contrast product to produce an image of the volumetric concentration of the contrast product in the breast.

19. The mammograph of claim 10, wherein the processing unit is further configured to determine a function linking the concentration of the product to the logarithms of the intensities of the at least one first image and the at least one second image.
